(19) United States
(12) Patent Application Publication
(10) Pub. No.: US 2011/0028809 A1
(43) Pub. Date: Feb. 3, 2011

(54) PATIENT MONITOR AMBIENT DISPLAY DEVICE

(75) Inventor: David Edward Goodman, Greenbrae, CA (US)

Correspondence Address: KNOBBE MARTENS OLSON & BEAR LLP, 2040 MAIN STREET, FOURTEENTH FLOOR, IRVINE, CA, 92614, US

(73) Assignee: MASIMO CORPORATION, Irvine, CA (US)

(21) Appl. No.: 12/845,607

(22) Filed: Jul. 28, 2010

Related U.S. Application Data
(60) Provisional application No. 61/229,633, filed on Jul. 29, 2009.

(51) Int. Cl. A61B 6/00

(52) U.S. Cl. 600/322; 600/476

(57) ABSTRACT

Embodiments of the disclosure include an orb or lamp communicating with a noninvasive monitor to provide a readily identifiable point indication of a wellness of a monitored patient. In an embodiment the orb emits a color gradient from a first color through at least two other colors responsive to values of a wellness measurement. Exemplary wellness indications include one or a statistical combination of blood constituent measurements, combinations of other physiological parameters, or the like.

### CROSS-REFERENCE TO RELATED APPLICATIONS

This application claims priority benefit under 35 U.S.C. §119(e) to U.S. Provisional Patent Application Ser. No. 61/229,633, filed Jul. 29, 2009, entitled “Patient Monitor Ambient Display Device.” The disclosure of which is incorporated in its entirety by reference herein.

### BACKGROUND

1. Field

The present disclosure relates to the field of patient monitors. More specifically, the disclosure relates to the display of various patient monitor characteristics.

2. Description of the Related Art

In order to assess patient condition, caregivers often desire knowledge of various physiological parameters of the patient. These physiological parameters include, for example, oxygen saturation (SpO₂), hemoglobin (Hb), blood pressure (BP), pulse rate (PR), perfusion index (PI), and Pleth Variable Index (PVI) and many others, or combinations of the same, or the like. This monitoring is important to a wide range of medical applications. Oximetry is one of the techniques that has been developed to accomplish the monitoring of some of these physiological characteristics. It was originally developed to study and to measure, among other things, the oxygen status of blood. Pulse oximetry—a noninvasive, widely accepted form of oximetry—relies on a sensor attached externally to a patient to output signals indicative of various physiological parameters, such as a patient's constituents or analytes, including, for example, those listed above as well as a percent value for carbon monoxide saturation (HbCO), methemoglobin saturation (HbMet), fractional saturations, total hematocrit, billirubins, others, or combinations of the same, or the like. As such a pulse oximeter is one of a variety of patient monitors that help provide monitoring of a patient's physiological characteristics.

A pulse oximeter sensor generally includes one or more energy emission devices, such as specific wavelength emitting LEDs, and one or more energy detection devices. The sensor generally attaches to a measurement site such as a patient's finger, toe, ear, ankle, or the like. An attachment mechanism positions the emitters and detector proximal to the measurement site such that the emitters project energy into the tissue, blood vessels, and capillaries of the measurement site, which in turn attenuate the energy. The detector then detects that attenuated energy. The detector communicates at least one signal indicative of the detected attenuated energy to a signal processing device such as an oximeter, generally through cabling attaching the sensor to the oximeter. The oximeter generally calculates, among other things, one or more physiological parameters of the measurement site.

Pulse oximeters are available from Masimo Corporation (“Masimo”) of Irvine, Calif. Moreover, some exemplary portable and other oximeters are disclosed in at least U.S. Pat. Nos. 6,770,028, 6,658,276, 6,157,850, 6,002,952, and 5,769,785, which are owned by Masimo and are incorporated by reference herein. Such oximeters have gained rapid acceptance in a wide variety of medical applications, including surgical wards, intensive care and neonatal units, general wards, home care, physical training, and virtually all types of monitoring scenarios.

Other noninvasive, minimally invasive, and invasive patient monitoring devices are similarly available to measure various parameters of a patient.

Typically, the physiological parameters are displayed to the caregiver as separate numbers, detailed graphs, and/or the like on a display of a patient monitor or the display of a multi-parameter patient monitor. Although this provides a large amount of data in a relatively small space, the greater the number of parameters being monitored, the more complicated and potentially cluttered a display may become.

### SUMMARY

There remains a need for a simple, highly visible, yet relatively unobtrusive indicator of individual patient parameters or a combination of parameters, such as for example, in an overall wellness assessment. This is particularly useful for parameters or combinations of parameters that are unlikely to change quickly. Such an indicator can provide quick information to a person desiring such information without requiring great concentration or effort in interpretation of a detailed display of graphical or numerical data. For example, it would be useful to provide a patient indicator outside a patient's room so that caregivers need not disturb sleeping patients, those with visitors, or the like when there is no need. A caregiver may also more quickly scan one such indicator or many to assess one or multiple patients. With indicators placed outside patients' rooms or in other conspicuous locations, a caregiver is also able to scan a hallway, department, or the like more efficiently to help locate patients in more urgent need of attention.

One aspect of the disclosure provides a patient monitor with a color-changing indicator that can display a status indication of, for example, hemoglobin, PVI, an overall state of wellness, combinations of the same, or other parameters, measurements or conditions of a patient. In an aspect of this disclosure, the color-changing indicator includes a wireless connection to the patient monitor for easy placement in a wide range of locations.

In an aspect of the disclosure, the color-changing indicator includes a soft glowing orb, cube, or other three-dimensional shape, to provide the patient indication to caregivers from multiple angles, allowing those not near or in line with a patient monitor screen to see the indication of the monitored parameter. In an aspect, the indicator may flash or otherwise change states to indicate alarm conditions as well.

### BRIEF DESCRIPTION OF THE DRAWINGS

The following drawings and the associated descriptions are provided to illustrate embodiments of the present disclosure and do not limit the scope of the claims. Corresponding numerals indicate corresponding parts, and the leading digit of each numbered item indicates the first figure in which an item is found.

FIG. 1 illustrates a perspective view of a patient monitor system in accordance with an embodiment of the disclosure.

FIG. 2 illustrates a block drawing of a patient monitor system such as the system of FIG. 1, in accordance with an embodiment of the disclosure.

FIG. 3 illustrates an exemplary color gradient as may be utilized by an embodiment of a patient monitor indicator device of the block diagram of FIG. 2 in accordance with an embodiment of the disclosure.

FIG. 4 illustrates a method of generating and displaying a non-alphanumeric visual indicator in accordance with an embodiment of the disclosure.

FIG. 5 illustrates another method of generating and displaying a non-alphanumeric visual indicator in accordance with an embodiment of the disclosure.

FIG. 6A illustrates a method of associating a visual indicator with a patient monitor in accordance with an embodiment of the disclosure.

FIG. 6B illustrates an exemplary screenshot of a patient monitor running a pairing software routine of FIG. 6A.

FIG. 7A illustrates a method of selecting a patient parameter for display through a visual indicator in accordance with an embodiment of the disclosure.

FIG. 7B illustrates an exemplary screenshot of a patient monitor running a parameter selection software routine of FIG. 7A.

### DETAILED DESCRIPTION

Aspects of the disclosure will now be set forth in detail with respect to the figures and various embodiments. One of skill in the art will appreciate, however, that other embodiments and configurations of the devices and methods disclosed herein will still fall within the scope of this disclosure even if not described in the same detail as some other embodiments. Aspects of various embodiments discussed do not limit the scope of the disclosure herein, which is instead defined by the claims following this description.

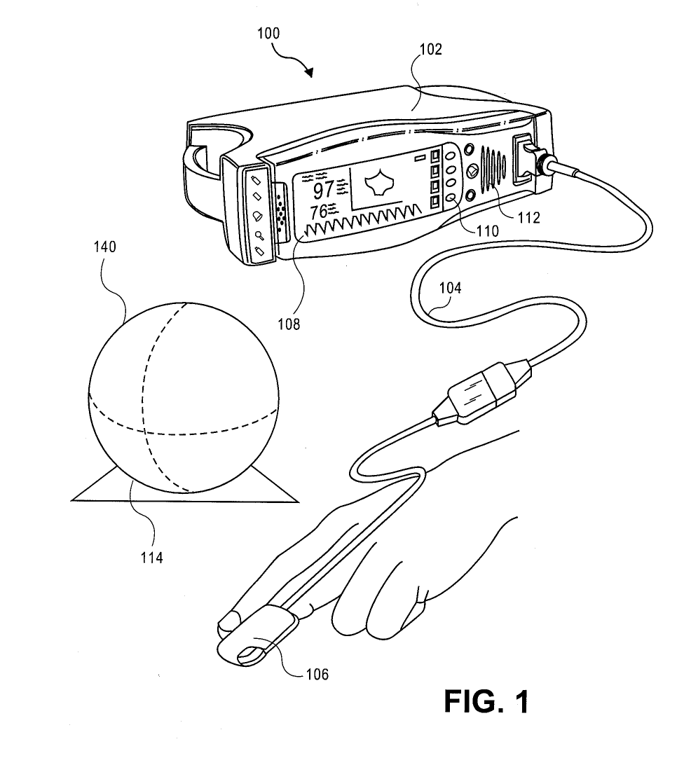

Turning to FIG. 1, an embodiment of a multi-parameter patient monitor system 100 is illustrated. The patient monitor system 100 includes a patient monitor 102 attached to a sensor 106 by a cable 104. The sensor 106 monitors various physiological data of a patient and outputs signals indicative of the parameters to the patient monitor 102 for processing. The patient monitor 102 generally includes a display 108, control buttons 110, and a speaker 112 for audible alerts. The display 108 is capable of displaying readings of various monitored patient parameters, which may include numerical readouts, graphical readouts, and the like. Display 108 may be a liquid crystal display (LCD), a cathode ray tube (CRT), a plasma screen, a Light Emitting Diode (LED) screen, Organic Light Emitting Diode (OLED) screen, or any other suitable display. A patient monitor system 102 may monitor oxygen saturation (SpO₂), perfusion index (PI), pulse rate (PR), hemoglobin count, and/or other parameters, weighted or other combinations of parameters, or the like. An embodiment of the patient monitoring system of FIG. 1, according to the present disclosure further includes an indicator 114 that provides non-textual indications of one or more patient parameters. In some embodiments, indicator 114 may be wired or wirelessly connected to the patient monitor 102, such as through a cable, radio frequency or IR transmissions, a public network, and/or a private network, which may include a LAN, W-LAN, WAN, cellular network, the Internet, combinations of the same, and/or the like.

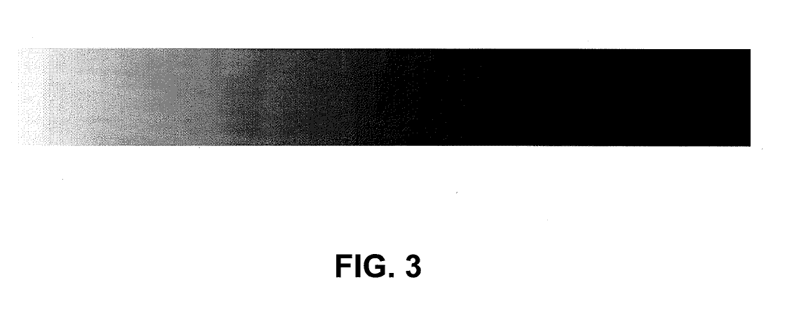

In an embodiment, indicator 114 comprises a glowing lamp 140 that may change colors to correspond to various parameter readings of the patient monitor. As used herein, one of skill in the art will understand that the term “lamp” refers to its broad and ordinary meaning known to an artisan and includes any of a wide variety of devices furnishing artificial light, such as by electricity or gas. A lamp may include one or more fluorescent or incandescent bulbs, LEDs, OLEDs, and/or the like. For example, the lamp 140 of indicator 114 may change slowly from green to yellow to red to indicate an overall wellness condition or other patient parameter as the monitor 102 processes signals from the sensor 106 representing a condition changing from a good to worsening state. In another embodiment, an indicator 114 may change from red to blue to indicate dropping oxygen saturation (SpO₂). Various shades of a single color can also provide indications of the state of a certain parameter or group of parameters. In an embodiment, the color change may follow a color gradient, a mathematical interpretation of a gradual change among two or more colors. For example, a linear gradient may specify a starting and ending color, with the colors at each point along the line specified by a mathematical function such as linear interpolation. FIG. 3 illustrates an exemplary color gradient between black and white. An artisan will recognize from the disclosure herein that the lamp 140 may include additional functionality to gain the attention of a caregiver under predetermined or user-defined behaviors of a monitored parameter, a group of monitored parameters combined by a weighted or other function or the like. For example, additional functionality may include blinking, sound generation, such as beeping, chirping, tones, or the like, message display, others, combinations of the same, or the like. Message displays may include patient room numbers, indications of which patient monitor is sending the parameter indications, and the like. In addition, a visual indicator 114 may further include components and functionality to allow alarm states to be transmitted to further devices, such as, for example, a caregiver's pager, mobile phone, PDA, computer, or the like.

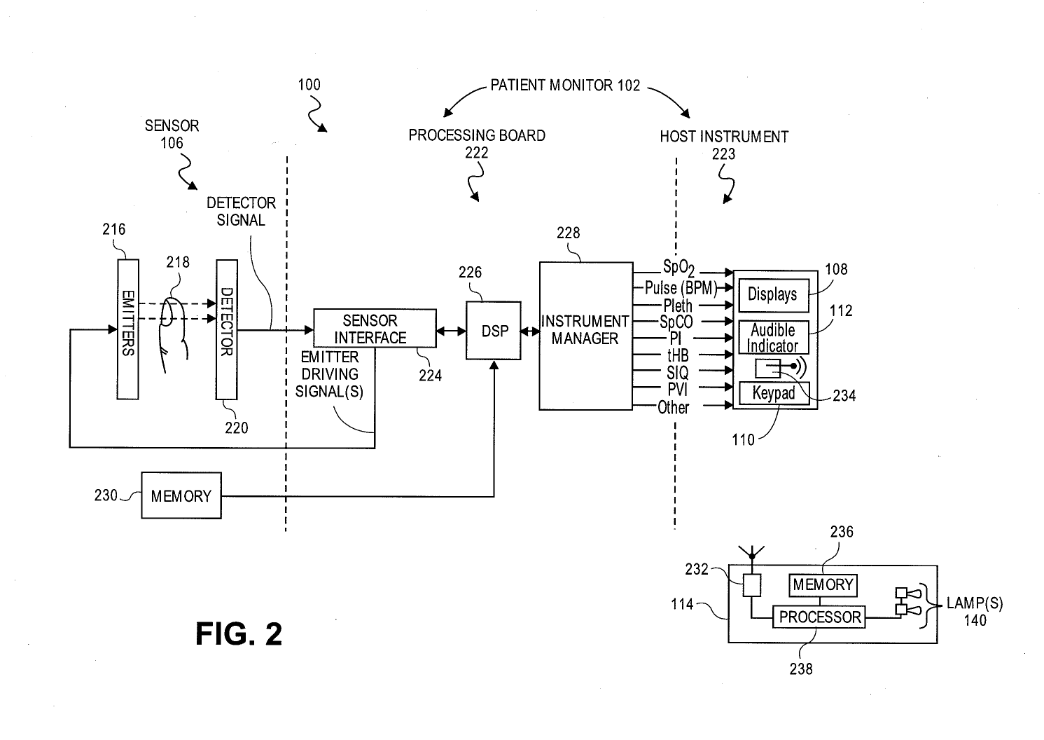

FIG. 2 illustrates exemplary details of an embodiment of the patient monitor system 100 in a block diagram format. Typically the sensor 106 includes energy emitters 216 located on one side of the patient monitoring site 218 and one or more detectors 220 located generally opposite. The patient monitoring site 218 is usually a patient's finger (as pictured), toe, ear lobe, or the like. Energy emitters 216, such as LEDs, emit particular wavelengths of energy through the flesh of a patient at the monitoring site 218, which attenuates the energy. The detector(s) 220 then detect the attenuated energy and send one or more representative signals to the patient monitor 102.

Specifically, an embodiment of the patient monitor 102 includes processing board 222 and a host instrument 223. In an exemplary embodiment, the processing board 222 includes a sensor interface 224, a digital signal processor (DSP) 226, and an instrument manager 228. The host instrument, which may be part of an assembled monitor 102 or part of a monitor where the board 222 and/or the instrument 223 are sources from different OEMs, the same OEM for different instrument branding of the like, may advantageously includes one or more displays 108, control buttons 110, a speaker 112 for audio messages, and a wireless signal broadcaster 234. Control buttons 110 may comprise a keypad, a full keyboard, a track wheel, and the like. Additionally embodiments of a patient monitor 102 can include buttons, switches, toggles, check boxes, and the like implemented in software and actuated by a mouse, trackball, touch screen, or other input device.

The sensor interface 224 receives the signals from the sensor 106 detector(s) 220 mat or may not condition the signals for noise and/or gain as desired and passes the signals to the DSP 226 for processing into measurements or indications of physiological parameters, combinations of physiological parameters, or the like. Some or all of the measurements can be transmitted to the instrument manager 228, which may further process the parameters for display by the host instrument 223. In some embodiments, the DSP 226 also communicates with a memory 230. In some embodiments, memory may be located on the sensor 106, in the cable 104, in the connector in the instrument, combinations of the foregoing, or the like. The memory 120 may store information related to the properties of the sensor that may be useful in processing the signals, such as, for example, emitter 216 energy wavelengths or other useful data. The elements of processing board 222 provide processing of the sensor 106 signals. Tracking or trending medical signals can prove challenging because the signals may include various anomalies that may not reflect an actual changing patient parameter. The processing board 222 processing generally attempts to filter limited duration anomalies, while preserving actual parameter behavior. The host instrument 223 then is able to display one or more physiological parameters according to instructions from the instrument manager 228.

In an embodiment of the present disclosure, the host instrument 223 also includes a wireless signal broadcaster 234, which it uses to send a signal to one or more visual indicators 114 indicative of an indicator display state for a particular patient status parameter or condition, for example, an overall wellness indication. Visual indicator 114, in an embodiment, includes an antenna 232, memory 236, a processor 238, and one or more lamps 140, such as one or more LEDs or other light sources. The antenna 232 can comprise a one-way or two-way antenna in various embodiments. The processor 232 may comprise a special or general purpose processor, a printed circuit board, or the like.

Antenna 232 receives signals broadcast by one of the board 222 or the host instrument 223 and the processor 238 interprets these signals to determine, for example, how to drive lamp 140. In an embodiment, these wireless signals may include encryption, a patient monitor identifier, or the like, so that each of multiple patient monitors can communicate with one or more specific visual indicators 114. In an embodiment, memory 236 may store one or more of an indication of which host instrument's signals to accept, unique indicator data or parameter selection information, display data, and/or the like. Memory 236 may include an EPROM, an EEPROM, RAM, and/or solid state memory, combinations of the same, or the like for example. The processor 238 can then utilize information from memory 236 to help interpret the received signals, such as by extracting information including a display color, display pattern (such as flashing), alarm conditions, patient location identifier, doctor identifier, paging information, caregiver, combinations of the same, and/or the like.

Memory 236 is preferably programmable so that a caregiver can leave an indicator 114 in one place and associate different patient monitors 102 with it at various times. In an embodiment, the memory 236 programming can be accomplished through a “pairing” function that links one or more patient monitors 102 with one or more visual indicators 114. In an embodiment, for example, a patient monitor 102 identifier can be stored in memory 236 for comparison with signals detected by antenna 232. Opposingly, a caregiver may move an indicator 114 or associate it with different stationary patient monitors 102 at various times. For example, a single indicator 114 may be placed at a nurse's station for a hospital department. A nurse, doctor, or other caregiver may then associate that indicator 114 with the patient monitor 102 that is monitoring critical patients in the department, or the like. One of skill in the art will understand, from the disclosure herein, that any of a wide variety of communications protocols could be used for the communications discussed. For example, a patient monitor system 100 can include Bluetooth® communications or other radio frequency communications protocols. In various embodiments, wireless signal broadcaster 234 and indicator antenna 232 can utilize one-way or two-way communications in transferring patient information for display.

In various embodiments, the patient monitor 102 can transmit signals approximately continuously, at periodic or random intervals, or whenever a change of a certain magnitude is detected, combinations of the same or the like. It is preferable for all or much of the signal processing to be done within the patient monitor 102, such as by processing board 222, to help allow the transmission of data to indicator 114 in relatively small packets. This helps limit the amount of processing required at the indicator 114. As an example, a transmission packet may include a number between 0 and a number X, between −X and +X, or the like to indicate the location along a color gradient spectrum to display. The information may also be a specific color code, drive current/voltage, duty cycle, alarm function, combinations of the same, or the like. The transmission packet may further include a patient monitor ID or other identifier as described herein allowing the indicator 114 to determine the proper signal to display. In an embodiment, the information may further include a display pattern or alarm state (such as, for example, solid or flashing), sound data for an included speaker, combinations of the same, or the like.

One example parameter that may be displayed by indicator 114 in this manner is the Pleth Variable Index (PVI) developed by Masimo Corporation, Irvine, California that is a measure of the dynamic changes in the perfusion index (PI) that occur during the respiratory cycle. In an embodiment, the measurement of PI can be defined as a ratio of pulsatile blood flow to nonpulsatile blood in peripheral tissue. In this embodiment, the PVI is a noninvasive measurement indicative of peripheral perfusion that can be continuously determined with a pulse oximeter.

If, for example, the PVI is chosen for display, in an embodiment, the instrument manager 228 creates a packet, including an indication of the color representative of the current PVI reading, for transmission to the visual indicator 114. The packet may further include an indication that the PVI is not in an alarm state. The wireless signal broadcaster 234 transmits the packet to visual indicator 114, which accepts the packet at antenna 232. Processor 238 can extracts the requisite information from the packet and causes lamp 140 to display the proper color as indicated by the current PVI readings. With a regular transmission of updated PVI readings, the lamp 140 will appear to change color with the changes in the PVI readings. The pace or acceleration of a color change provides a strong visual indication to a caregiver of the changes in PVI without a need to see and interpret specific alphanumeric characters or remember what previous readings had been. For example, a changing color from green to red can give a quick indication of a drop in a patient parameter, whereas a caregiver who has not been monitoring that parameter may not understand what a single numeric reading means or would have to recall what the relationship was between the current reading and previous readings. Similarly, the lamp 140 may flash or provide other indications of an alarm state to attract quick attention from a caregiver.

The indicator 114 can also display other blood constituents, combinations of other parameters, or the like as other examples. Parameters that are likely to change relatively slowly are ideally suited to a color gradient or similar visual indicator 114, but other parameters can also be suitably displayed.

An indicator 114 as disclosed herein can be advantageous for a number of situations. As explained, it can often be seen from a greater distance, and a caregiver does not need to be facing a patient monitor display 108 to derive an understanding or even a glimpse of a patient's condition. For example, an embodiment of the present disclosure may be particularly useful in an operating room, such that a surgeon can obtain a quick understanding of a PI reading for a patient by looking at a visual indicator 114 rather than having to ask someone else in the room or distracting his or her attention by trying to read a detailed alphanumeric display of a patient monitor. Similarly, indications of the depth of anesthesia can be calculated from a noninvasive monitor. In an embodiment, a surgeon can obtain a quick understanding of this measurement without asking the anesthesiologist who may be attending to other aspects of the patient's care. Similarly, it may be more important for the anesthesiologist to monitor the main display 108 of the patient monitor, and the display may be turned away from the surgeon interested in a general status indication.

Another feature of embodiments of the disclosure is that the visual indicator 114 may be more harmonious with the environment. In a crowded hospital setting, aural alerts or alarms can be intrusive to patient care or caregiver situations. Multiple such alerts and alarms can simply become noise over time. Additionally, aural alerts may at times frighten a patient unnecessarily, creating stress and anxiety that may be counterproductive to the patient's treatment. In an embodiment, a soft glowing indicator 114 can be more soothing to a patient, while still conveying important information to a caregiver.

The visual indicator 114 has been discussed mainly in terms of a wireless embodiment. This feature can provide greater flexibility in placement of the indicator 114. For example, an indicator can be placed on top of a patient monitor 102, beside a patient's bed, outside a patient's room, such as near the door to the room or in a central area, such as a nurse's station, attached to an IV tree, or the like, with less intrusion from wiring. However, other embodiments of the patient monitor system 100 with indicator 114 may include wired connections between host instrument 223 and indicator 114, integration between host instrument 223 and indicator 114, or the like. In an embodiment, indicator 114 includes a power source, such as a battery, solar charger, or the like, to further increase the placement options for indicator 114. In other embodiments, indicator 114 may be adapted to connect to a power outlet, a USB or mini-USB port or the like. Additionally, while the figures illustrate the indicator 114 as comprising a generally spherical lamp 140, other embodiments may include varying shapes, comprise single or multiple lamps as a strip, cube, or the like.

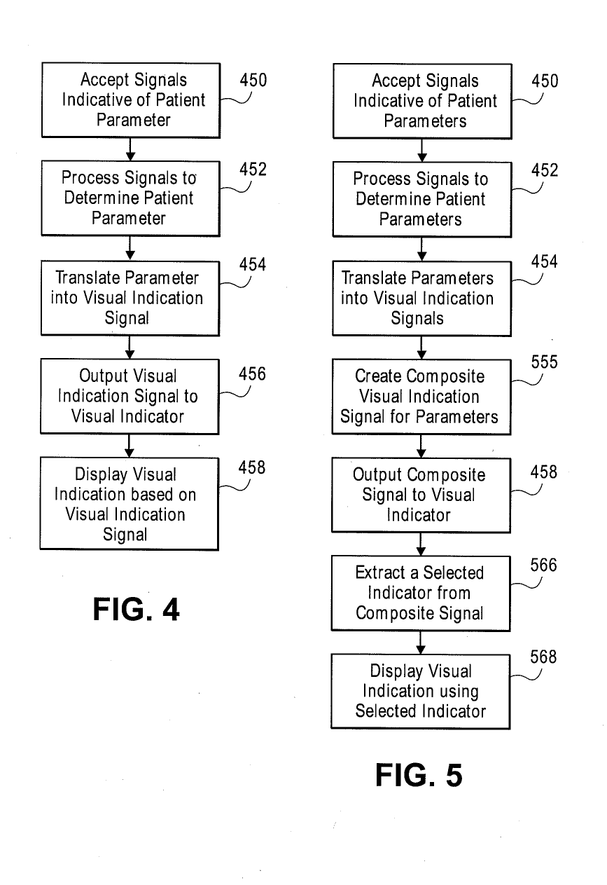

Although referenced generally herein, a number of exemplary methods will now be more specifically discussed with reference to FIGS. 4-7B. FIG. 4 illustrates an exemplary method for interpreting patient parameters and creating non-alphanumeric display indications. A patient monitor 102 accepts signals indicative of one or more patient parameters (block 450). In an embodiment, the patient monitor processes these signals to determine one or more patient parameter measurements (block 452). In an embodiment, this may also include checking the one or more patient parameter measurements for anomalies. For example, a given parameter may fall within a certain range or only be able to change a certain amount from a prior reading. If either of these or another similar rule is broken, the patient monitor may regard the parameter reading as an anomaly and take remedial measures including statistically combining previous data, adjusting emphasis to previous data, interpolating from more trustworthy data, combinations of the same, or the like.

In block 454, valid patient parameter measurements are translated into data expected by visual indicator 114. There are a number of possible options for this translation. The translation may include interpretation of a continuum between two or more colors, and a linear interpolation of a color along the color gradient between these colors. In one embodiment, the visual indication signal may include a numerical value representative of a distance along a color gradient, a code representative of a specific color to be displayed, the actual parameter measurement (for interpretation within a visual indicator processor 238, for example), and/or the like. In an embodiment, the visual indication signal may further include a display code to indicate how the color should be displayed, such as solid, pulsing, flashing, the brightness level, combinations of the same, or the like. In an embodiment, the visual indication signal may further include a source or destination code, such as, for example, a patient monitor 102 identifier or a visual indicator 114 identifier, to help determine what visual indicator is the intended receiver. Such an identifying code is generally necessary when the visual indicator is connected wirelessly or through an addressable computer network, for example. The patient monitor 102 outputs the visual indication signal to the indicator 114 at block 456. The visual indicator 114 receives the signal, decodes it, and alters the one or more lamps 140 accordingly at block 458.

FIG. 5 illustrates another embodiment of a method for generating substantially or at least partially non-alphanumeric display indications of patient parameters. In this method, the patient monitor 102 accepts signals indicative of multiple patient parameters and interprets them in much the same way as described with respect to FIG. 4 (blocks 450-54). However, when, for example, a patient monitor creates visual indication codes for each of a number of parameters, the patient monitor creates a signal packet that includes the visual indications for each parameter (block 555). For example, the signal may include five bytes of data, with the first relating to SpO₂, the second relating to PVI, the third to an overall wellness condition, the fourth to a state of anesthesia, and the fifth being the patient monitor identifier. The signal packet is output to the visual indicator 114 (block 456), which, in an embodiment, is accomplished in much the same way as sending signals within individual parameters. As illustrated in FIG. 5, the visual indicator 114 extracts the source or destination identifier from the signal packet to ensure it is processing the proper signal. It can extract the desired data from the signal packet (such as the third byte relating to wellness, in the example) (block 566), and alter the lamp(s) 140 according to the extracted data (block 568). In such an embodiment, a switch, button, or other selection feature may be incorporated within visual indicator 114 to choose which parameter should be displayed. Memory 236 of indicator 114 may also store data regarding which parameter should be extracted and displayed.

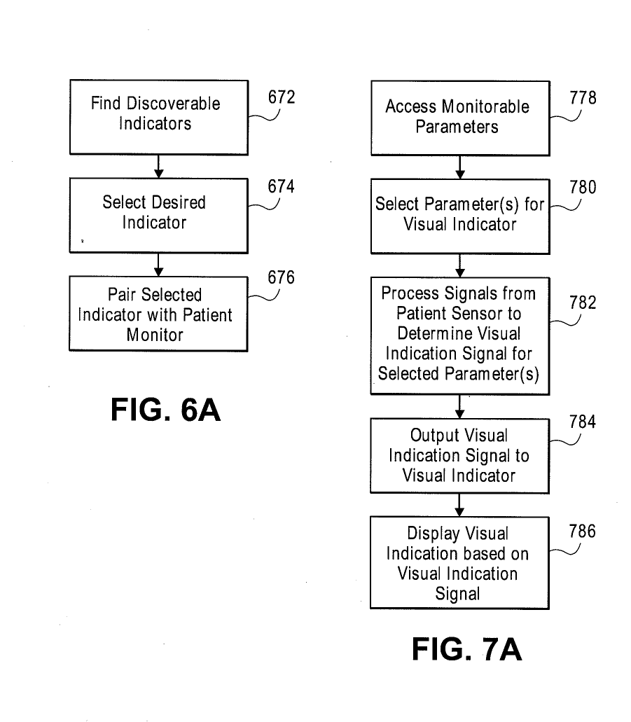

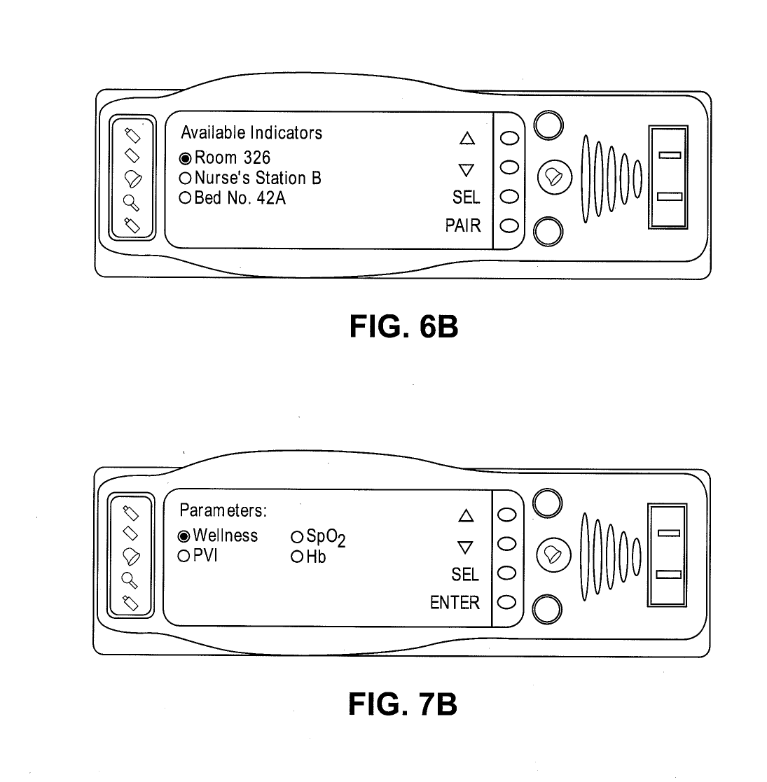

FIG. 6A illustrates an exemplary method of associating a patient monitor 102 and a visual indicator 114, which may be referred to as “pairing.” In an embodiment, a user initiates a discoverable state for the indicator 114. This may comprise powering on the indicator 114, switching the indicator to a special discovery state, and/or the like. In a discovery state, for example, the antenna 232 of indicator 114 may broadcast the indicator's presence and/or availability to pair. The patient monitor 102 finds the discoverable indicators 114 (block 672). In an embodiment, such a function has the patient monitor 102 accept broadcast signals from visual indicators 114 that are within range. The user can then select among one or more possible visual indicators, such s through a selection screen on the patient monitor 102 (block 674). FIG. 6B illustrates an exemplary patient monitor display in this state. Available indicators may appear with names, numbers, or other identifying characteristics, and one may be selectable by a radio button or the like. Once the visual indicator 114 is selected, the patient monitor 102 and/or the selected visual indicator 114 can store information identifying the signals to send and/or receive during normal operation, thus linking the patient monitor 102 and visual indicator 114 together (block 676). In other embodiments, pairing may occur through a cable connection, whether or not the visual indicator 114 will remain connected to the patient monitor 102 during normal operation and display of patient parameter indications.

Apart from selecting which patient monitor 102 and which visual indicator 114 should be paired, a user, in an embodiment, may also be able to select which patient parameter should be displayed with the visual indicator 114. FIG. 7A illustrates an exemplary method for achieving this. A patient monitor 102 may include a software routine to aid in this selection, in an embodiment. The user accesses this software routine, which retrieves the parameters that can be monitored based on, for example, the attached sensor(s) (block 778). FIG. 7B illustrates an exemplary screen of patient monitor 102 running the parameter selection routine. In an embodiment, each monitorable parameter that can be translated into a non-alphanumeric visual indicator will be listed, such as through a set of radio buttons. The user can choose one parameter to be displayed by a visual indicator (block 780). In an embodiment, a patient monitor 102 may pair with multiple visual indicators, and each one may display a parameter indication. Once selected, the signals from the sensor(s) 106 can be interpreted, output to the visual indicator, and displayed according to methods similar to those described in FIGS. 4 and 5 (blocks 782-86). In other embodiments, similar selection of parameters may occur at a visual indicator 114, such as through software, physical buttons or switches, or the like.

In additional embodiments, multiple parameters may be selected simultaneously, such as through the use of check boxes rather than the radio buttons illustrated in FIG. 7B. If multiple parameters are selected, the patient monitor 102, the processor 238 of visual indicator 114, or a combination of the two may process the selected patient parameter readings into a more generalized wellness indication for display by the lamp(s) 140. It is further contemplated that different aspects of the visual indicator 114 can coincide with different ones of the selected parameters. For example, a caregiver may select both SpO₂ and pulse rate for monitoring. In one embodiment, the two are combined into a wellness indication with, for example, a generally good SpO₂/pulse rate combination being displayed toward the green end of the spectrum and a generally bad reading being displayed a red end of the spectrum. In another embodiment, the different parameters may remain at least partially distinguishable, however. For example, SpO₂ may be represented by the color and pulse rate may be represented by brightness of the lamp, allowing a caregiver to more readily distinguish between a deteriorating patient condition because of a slowing heart rate or due to a lower oxygen saturation. Similarly, SpO₂ and pulse rate may use multiple colors to distinguish among high and low readings for each parameter. For example, a high SpO₂ and a high pulse rate may be indicated by a more red color to the lamp(s) 140; a green color may indicate a high SpO₂ but a low pulse rate; a blue color may indicate a low SpO₂ and a low pulse rate; and a yellow color may indicate a low SpO₂ and high pulse rate. Skilled artisans will understand similar combinations of parameters may be displayed through other combinations of colors, visual indications, such as pulses, audio indications, and the like.

As described generally herein, in an embodiment, the patient monitor 102 is programmable to determine which signals to transmit or broadcast to an indicator 114. In an embodiment, for example, patient monitor 102 comprises software to allow a caregiver to review various indicator display options on display 108 and select among options using keypad 110 or other input methods. For example, a caregiver may select to have the indicator display SpO₂, a general wellness indication, PVI, others, combinations of the same, or the like. In this manner, a caregiver can select the most important parameter that he or she wishes to monitor. For example, a caregiver may determine that a particular patient's condition is likely to manifest the patient's worsening state first or most dramatically through a change in SpO₂. The caregiver may thus wish to have quick access to changes in that parameter and select to have that parameter displayed by the visual indicator 114. Other patients may simply be generally monitored through a general wellness indication that amalgamates multiple parameters.

In an embodiment, a caregiver can also select or alter other options for the visual indicator 114, such as for example, brightness, color selections, alarm conditions, alarm settings (such as an audible volume or tone or visual flashing, for example), and the like, for lamp 140. In other embodiments, some or all of these features may be selectable through controls accessible through indicator 114. For example, in an embodiment, a patient monitor 102 may use wireless signal broadcaster 234 to send indications of multiple parameters. The indicator's processor 238 may then select among these various condition indications to display the patient indication desired by the caregiver. Similarly, in an embodiment, indicator 114 may include controls to vary the colors, brightness, or other aspects of lamp 140. In another embodiment, processor 238 and/or memory 236 may be programmable through a PC, server, handheld device, or the like through a wired or wireless connection. For example, in an embodiment, an indicator 114 may utilize a mini-USB connection to connect to a power adaptor as well as a laptop, handheld device, smart phone, or the like to access indicator 114 options.

Although the foregoing has been described in terms of certain specific embodiments, other embodiments will be apparent to those of ordinary skill in the art from the disclosure herein. Moreover, the described embodiments have been presented by way of example only, and are not intended to limit the scope of the disclosure. Indeed, the novel methods and systems described herein may be embodied in a variety of other forms without departing from the spirit thereof. Accordingly, other combinations, omissions, substitutions, and modifications will be apparent to the skilled artisan in view of the disclosure herein. For example, various functions described as occurring at one or more element of the patient monitor 102 may also or alternatively be accomplished within indicator 114 or vice versa. Thus, the present disclosure is not limited by the preferred embodiments, but is defined by reference to the appended claims. The accompanying claims and their equivalents are intended to cover forms or modifications as would fall within the scope and spirit of the disclosure.

What is claimed is:

1. A patient wellness indicator configured to provide a non-numeric indication of patient wellness in at least a substantial portion of a display element, the indicator comprising:
a stand-alone lamp device configured to alter its color in response to a received indication of one or more physiological parameters or combination of physiological parameters measured by a noninvasive optical sensor and processed through a patient monitor, the lamp device capable of incrementally emitting at least three colors, each increment corresponding to a change in said received indication, said incremental changes in one direction corresponding to said received indication approaching a increasing wellness, said incremental changes in another direction corresponding to said received indication approaching a decreasing wellness, said lamp device also configured to emit at least one alarm condition where a decreasing wellness falls below a threshold where a caregiver should administer care;
a memory capable of storing a plurality of said received indications; and
a processor capable of accessing said memory and driving said lamp device to emit said colors, said processor capable of statistically combining said received indications to control said emission color of said lamp device according to a predetermined rate of change.

2. The indicator of claim 1 wherein at least three colors comprises a color gradient from a first of said at least three colors, through a second of said at least three colors, to a third of said at least three colors.

3. The indicator of claim 2 wherein said at least three colors include substantially red.

4. The indicator of claim 2 wherein said at least three colors include substantially yellow.

5. The indicator of claim 2 wherein said at least three colors include substantially green.

6. The indicator of claim 2 wherein said at least three colors include substantially blue.

7. The indicator of claim 2 wherein said at least three colors include substantially three or more of red, yellow, green, orange and blue.

8. The indicator of claim 1 comprising a communication module configured to receive said received indications.

9. A patient monitoring device capable of providing a single point indication of wellness, the device comprising:
a memory storing a plurality of wellness measurements received from a processing device configured to process optical attenuation information from a noninvasive optical sensor;
a processor capable of reading said measurements from said memory and outputting information to be displayed; and
a display including a first display area and a second display are, said first display area configured to display a color gradient responsive to said wellness measurements, said first display area substantially larger than said second display area when said device is in a wellness display mode.

10. The device of claim 9 wherein said first display area displays said color gradient as including a rising bar.

11. The device of claim 9 wherein said first display area displays said color gradient as including a substantially spherical or circular shape.

12. The device of claim 9 wherein said first display area displays said color gradient as including a horizontal growing bar.

13. The device of claim 9 wherein said first display area displays said color gradient as including a flashing element.

14. The device of claim 9 comprising a transducer producing a sound alarm when said wellness measurements exceed a threshold.

15. The device of claim 9 wherein the patient parameters include at least one of PVI, one or more blood constituents, and depth of anesthesia.

16. A method of monitoring a patient comprising:
providing a patient status indicator including a lamp, the lamp visible from a plurality of angles;
accepting at least one patient parameter from a patient monitor, wherein the at least one patient parameter is indicative of at least one patient health characteristic sensed by the patient monitor;
translating the at least one patient parameter into a single color indicator of patient status; and
displaying the single color indicator using the lamp.

17. The method of claim 16 wherein the lamp is provided in a conspicuous location outside the patient's room.

18. The method of claim 16 further comprising the step of:
associating the patient status indicator with the patient monitor.

19. The method of claim 18 wherein patient parameters are not accepted from unassociated patient monitors.

20. The method of claim 16 wherein the signal is indicative of multiple patient parameters and the translating step comprises combining the multiple patient parameters into a single wellness parameter.

21. The method of claim 16 wherein the steps of detecting, determining, translating, and displaying are repeated over time and the single color indicator changes hue gradually over time along a color gradient continuum in relation to the at least one patient parameter.
